(19) United States
(12) Patent Application Publication
(10) Pub. No.: US 2024/0425922 A1
(43) Pub. Date: Dec. 26, 2024

(54) METHODS FOR THE TREATMENT OF TRAUMATIC BRAIN INJURY

(71) Applicant: VDS723, LLC, Scottsdale, AZ (US)

(72) Inventor: Divyen Patel, Schoharie, NY (US)

(21) Appl. No.: 18/635,936

(22) Filed: Apr. 15, 2024

Related U.S. Application Data
(60) Provisional application No. 63/460,019, filed on Apr. 17, 2023.

(51) Int. Cl. C12Q 1/6883

(52) U.S. Cl. CPC C12Q 1/6883; C12Q 2600/158; C12Q 2600/178

(57) ABSTRACT

Provided herein is a biomarker-based approach provides an accurate diagnosis of mild traumatic brain injury (mTBI) or traumatic brain injury (TBI), and allows a clinician to distinguish between TBI or mTBI and PTSD. A plurality of miRNAs are isolated from a patient sample and anayzed using Applicant's disclosed method.

### CROSS-REFERENCE TO RELATED APPLICATIONS

This application claims the benefit of and priority to U.S. Provisional Application No. 63/460,019, filed Apr. 17, 2023, the contents of which are incorporated by reference in its entirety.

### BACKGROUND

Mild traumatic brain injury (mTBI) affects approximately 20% of deployed U.S. service members 500,000 service members and veterans). 15-30% of veterans experience persistent neurobehavioral impairments for months or years, including cognitive dysfunction, sleep disruption, emotional distress, and chronic pain. For many this leads to addiction and suicide.

The 2017 CDC Youth Risk Behavior Survey found that 15.1% of students reported having at least one concussion related to sports or physical activity, and 6.0% reported having two or more. Playing on more than one sports team was found to further increase the risk for concussion.

This disclosure provides a fast and accurate process to identify TBI and provide clinicians with important information to treat TBI, including mTBI.

### SUMMARY

Applicant's process takes advantage of global miRNA profiles as diagnostic indicators of complex neuropathological differences between m traumatic brain injury (mTBI) or Post Traumatic Stress Disorder (PTSD) conditions. mTBI affects approximately 20% of deployed U.S. service members, which translates into more than 500,000 service members and veterans. Of those impacted soldiers, 15% to 30% experience persistent neurobehavioral impairments for months or years, including cognitive dysfunction, sleep disruption, emotional distress, depression, and chronic pain. The current lack of easily accessible and accurate diagnostic biomarkers for mTBI often impedes the timely application of appropriate therapy and the re-integration of affected veterans and civilians affected by TBI back into normal civilian life. In addition, a significant overlap of mTBI symptoms with those of post-traumatic stress disorder (PTSD) further complicates accurate mTBI diagnosis.

Applicant's biomarker-based approach provides an accurate diagnosis of mTBI and TBI, and allows a clinician to distinguish between mTBI and PTSD. The method is relatively inexpensive, portable, and minimally invasive. Application of this test can can improve outcome predictions and enable individualized treatments for our veterans and other Americans with mTBI.

Applicant's method utilizes global miRNA profiles as diagnostic indicators of complex pathological changes and of immune responses that reflect neuropathological conditions. The method can also be used to monitoring progress of therapies both short and long term.

Thus, in one aspect, provided herein is a method for selecting a patient for traumatic brain injury (TBI) therapy comprising, or consisting essentially of, or yet further consisting of, analyzing a plurality of miRNA isolated from a biological sample isolated from the patient by a method known in the art or as disclosed herein. The method can include maintaining a classification model configured to generate an output which is a function of expression levels of the plurality of miRNA, one or more clinical factors, and one or more offsets or weights. The method may include receiving data indicative of expression levels of the plurality of miRNA from a plurality of respective probes of a polymerase chain reaction (PCR) test of the patient. The method may include computing a first score as an output from the classification model, based on the one or more clinical factors for the patient and the expression levels of the plurality of miRNA in the biological sample. The method may include computing a second score based on the first score, the second score being within a range of values. When the second score satisfies a threshold value within the range, the patient may be selected for the TBI therapy.

In some embodiments, the one or more clinical factors include genomic gender, genomic post-traumatic stress disorder (PTSD) status, genomic time from event, and age. In some embodiments, the classification model may include a support vector machine with a slope being a coefficient, an offset as the intercept, and dependent variables as values for the one or more clinical values and expression levels of the subset of miRNAs. In some embodiments, the second score is computed as a sigmoid function of the first score from the classification model. In some embodiments, the method may include training the classification model using a training dataset, to determine the one or more offsets or weights. In some embodiments, the plurality of respective probes are configured to measure expression levels of the plurality of miRNA. The plurality of miRNA may include U8, U58B, U27, U83A, HBII-289, U55, HBII-239, U38B, U56, U15B, U35A, ACA48, U91, ENSG000001941, hsa-miR-671-5p, hcmv-miR-US4, hsa-miR1285, or hsa-miR455-3p.

Also provided is a method for selecting a patient for post traumatic stress disorder therapy (PTDS) therapy comprising, or consisting essentially of, or yet further consisting of, analyzing a plurality of miRNA isolated from a biological sample isolated from the patient by a method known in the art. The method may include maintaining a classification model configured to generate an output which is a function of expression levels of the plurality of miRNA, one or more clinical factors, and one or more offsets or weights. The method may include receiving data indicative of expression levels of the plurality of miRNA from a plurality of respective probes of a polymerase chain reaction (PCR) test of the patient. The method may include computing a first score as an output from the classification model, based on the one or more clinical factors for the patient and the expression levels of the plurality of miRNA in the biological sample. The method may include computing a second score based on the first score, the second score being within a range of values. When the second score satisfies a threshold value within the range, the patient may be selected for the PTSD therapy.

In one embodiment, a method for determining if a patient is suffering from TBI or PTDS is provided, the method comprising, or consisting essentially of, or yet further consisting of, analyzing a plurality of miRNA isolated from a biological sample isolated from the patient by a method. The method may include maintaining a classification model configured to generate an output which is a function of expression levels of the plurality of miRNA, one or more clinical factors, and one or more offsets or weights. The method may include receiving data indicative of expression levels of the plurality of miRNA from a plurality of respective probes of a polymerase chain reaction (PCR) test of the patient. The method may include computing a first score as an output from the classification model, based on the one or more clinical factors for the patient and the expression levels of the plurality of miRNA in the biological sample. The method may include computing a second score based on the first score, the second score being within a range of values. A score greater than a threshold may identify the patient is suffering from TBI and a score less than the threshold identifies the patient as suffering from PTSD.

With respect to the above and disclosed methods, the patient is a human patient. In another aspect the patient is suffering from symptoms of PTSD.

In a separate embodiment of the above methods, the biological sample comprises peripheral blood mononuclear cells.

### DETAILED DESCRIPTION

Throughout this disclosure, various publications, patents and published patent specifications are referenced by an identifying citation. The disclosures of these publications, patents and published patent specifications are hereby incorporated by reference into the present disclosure to more fully describe the state of the art to which this disclosure pertains.

As used herein, certain terms may have the following defined meanings. As used in the specification and claims, the singular form “a,” “an” and “the” include singular and plural references unless the context clearly dictates otherwise. For example, the term “a cell” includes a single cell as well as a plurality of cells, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about.” The term “about” also includes the exact value “X” in addition to minor increments of “X” such as “X+0.1” or “X−0.1.” It also is to be understood, although not always explicitly stated, that the reagents described herein are merely exemplary and that equivalents of such are known in the art.

The practice of the present technology will employ, unless otherwise indicated, conventional techniques of organic chemistry, pharmacology, immunology, molecular biology, microbiology, cell biology and recombinant DNA, which are within the skill of the art. See, e.g., Sambrook, Fritsch and Maniatis, Molecular Cloning: A Laboratory Manual, 2^{nd} edition (1989); Current Protocols In Molecular Biology (F. M. Ausubel, et al. eds., (1987)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, a Laboratory Manual, and Animal Cell Culture (R. I. Freshney, ed. (1987)).

The term “treating” as used herein is intended to encompass curing as well as ameliorating at least one symptom of the condition or disease such as TBI, mTBI or PTSD.

“An effective amount” or “therapeutically effect amount” intends to indicate the amount of a compound or agent administered or delivered to the patient which is most likely to result in the desired response to treatment.

A “patient” as used herein intends an animal patient, a mammal patient or yet further a human patient. For the purpose of illustration only, a mammal includes but is not limited to a simian, a murine, a bovine, an equine, a porcine or an ovine subject.

The term “clinical outcome”, “clinical parameter”, “clinical response”, or “clinical endpoint” refers to any clinical observation or measurement relating to a patient's reaction to a therapy. Non-limiting examples of clinical outcomes include alleviation of symptoms or clinical symptoms of mTBI, TBI or PTSD.

The term “identify” or “identifying” is to associate or affiliate a patient closely to a group or population of patients who likely experience the same or a similar clinical response to a therapy.

The term “selecting” a patient for a therapy refers to making an indication that the selected patient is suitable for the therapy. Such an indication can be made in writing by, for instance, a handwritten prescription or a computerized report making the corresponding prescription or recommendation.

“Detecting” as used herein refers to determining the presence of a nucleic acid of interest in a sample or the presence of a protein of interest in a sample. Detection does not require the method to provide 100% sensitivity and/or 100% specificity.

“Detectable label” as used herein refers to a molecule or a compound or a group of molecules or a group of compounds used to identify a nucleic acid or protein of interest. In some cases, the detectable label can be detected directly. In other cases, the detectable label can be a part of a binding pair, which can then be subsequently detected. Signals from the detectable label can be detected by various means and will depend on the nature of the detectable label. Detectable labels can be isotopes, fluorescent moieties, colored substances, and the like. Examples of means to detect detectable label include but are not limited to spectroscopic, photochemical, biochemical, immunochemical, electromagnetic, radiochemical, or chemical means, such as fluorescence, chemifluorescence, or chemiluminescence, or any other appropriate means.

“TaqMan® PCR detection system” as used herein refers to a method for real time PCR. In this method, a TaqMan® probe which hybridizes to the nucleic acid segment amplified is included in the PCR reaction mix. The TaqMan® probe comprises a donor and a quencher fluorophore on either end of the probe and in close enough proximity to each other so that the fluorescence of the donor is taken up by the quencher. However, when the probe hybridizes to the amplified segment, the 5′-exonuclease activity of the Taq polymerase cleaves the probe thereby allowing the donor fluorophore to emit fluorescence which can be detected.

As used herein, the term “sample” or “test sample” refers to any liquid or solid material containing nucleic acids such as miRNA, e.g., peripheral blood mononuclear cells (PBMCs). In suitable embodiments, a test sample is obtained from a biological source (i.e., a “biological sample”), such as cells in culture or a tissue sample from an animal, preferably, a human. In an exemplary embodiment, the sample is a biopsy sample.

Traumatic Brain Injury or “TBI”, a form of acquired brain injury, occurs when a sudden trauma causes damage to the brain. TBI can result when the head suddenly and violently hits an object or when an object pierces the skull and enters brain tissue. Symptoms of a TBI can be mild, moderate, or severe, depending on the extent of the damage to the brain. A person with a mild TBI may remain conscious or may experience a loss of consciousness for a few seconds or minutes. Other symptoms of mild TBI include headache, confusion, lightheadedness, dizziness, blurred vision or tired eyes, ringing in the ears, bad taste in the mouth, fatigue or lethargy, a change in sleep patterns, behavioral or mood changes, and trouble with memory, concentration, attention, or thinking. A person with a moderate or severe TBI may show these same symptoms, but may also have a headache that gets worse or does not go away, repeated vomiting or nausea, convulsions or seizures, an inability to awaken from sleep, dilation of one or both pupils of the eyes, slurred speech, weakness or numbness in the extremities, loss of coordination, and increased confusion, restlessness, or agitation. See https://www.ncbi.nlm.nih.gov/books/NBK542588/for definitions of this injury, last accessed on Apr. 6, 2023.

Mild Traumatic Brain Injury or “mTMI” is an injury that causes physiological disruption of brain function, as manifested by at least one of the following: 1. any period of loss of consciousness; 2. any loss of memory for events immediately before or after the accident; 3. any alteration in mental state at the time of the accident (eg, feeling dazed, disoriented, or confused); and 4. focal neurological deficit(s) that may or may not be transient; but where the severity of the injury does not exceed the following: loss of consciousness of approximately 30 minutes or less; after 30 minutes, an initial Glasgow Coma Scale (GCS) of 13-15; and posttraumatic amnesia (PTA) not greater than 24 hours. See https://acrm.org/wp-content/uploads/pdf/TBIDef_English_10-10.pdf, last accessed on Apr. 6, 2023.

It is generally understood (see, e.g., https://www.mayoclinic.org/diseases-conditions/traumatic-brain-injury/diagnosis-treatment/drc-20378561, last accessed on Apr. 6, 2023), that mild traumatic brain injuries usually require no treatment other than rest and over-the-counter pain relievers to treat a headache. However, a person with a mild traumatic brain injury usually needs to be monitored closely at home for any persistent, worsening or new symptoms. He or she may also have follow-up doctor appointments. The disclosed methods can be used to monitor a patient and his or her recovery. https://www.mayoclinic.org/diseases-conditions/traumatic-brain-injury/diagnosis-treatment/drc-20378561

Emergency care for moderate to severe traumatic brain injuries focuses on making sure the person has enough oxygen and an adequate blood supply, maintaining blood pressure, and preventing any further injury to the head or neck. People with severe injuries may also have other injuries that need to be addressed. Additional treatments in the emergency room or intensive care unit of a hospital will focus on minimizing secondary damage due to inflammation, bleeding or reduced oxygen supply to the brain.

Medications to limit secondary damage to the brain immediately after an injury may include: Anti-seizure drugs. People who've had a moderate to severe traumatic brain injury are at risk of having seizures during the first week after their injury. An anti-seizure drug may be given during the first week to avoid any additional brain damage that might be caused by a seizure. Continued anti-seizure treatments are used only if seizures occur. Coma-inducing drugs. Doctors sometimes use drugs to put people into temporary comas because a comatose brain needs less oxygen to function. This is especially helpful if blood vessels, compressed by increased pressure in the brain, are unable to supply brain cells with normal amounts of nutrients and oxygen. Diuretics. These drugs reduce the amount of fluid in tissues and increase urine output. Diuretics, given intravenously to people with traumatic brain injury, help reduce pressure inside the brain. Surgery Emergency surgery may be needed to minimize additional damage to brain tissues. Surgery may be used to address the following problems: Removing clotted blood (hematomas). Bleeding outside or within the brain can result in a collection of clotted blood (hematoma) that puts pressure on the brain and damages brain tissue. Repairing skull fractures. Surgery may be needed to repair severe skull fractures or to remove pieces of skull in the brain. Bleeding in the brain. Head injuries that cause bleeding in the brain may need surgery to stop the bleeding. Opening a window in the skull. Surgery may be used to relieve pressure inside the skull by draining accumulated cerebrospinal fluid or creating a window in the skull that provides more room for swollen tissues.

Most people who have had a significant brain injury will require rehabilitation. They may need to relearn basic skills, such as walking or talking. The goal is to improve their abilities to perform daily activities. Therapy usually begins in the hospital and continues at an inpatient rehabilitation unit, a residential treatment facility or through outpatient services. The type and duration of rehabilitation is different for everyone, depending on the severity of the brain injury and what part of the brain was injured.

Post Traumatic Stress Disorder or “PTSD” Posttraumatic stress disorder (PTSD) is a psychiatric disorder that may occur in people who have experienced or witnessed a traumatic event, series of events or set of circumstances. An individual may experience this as emotionally or physically harmful or life-threatening and may affect mental, physical, social, and/or spiritual well-being. Examples include natural disasters, serious accidents, terrorist acts, war/combat, rape/sexual assault, historical trauma, intimate partner violence and bullying. People with PTSD have intense, disturbing thoughts and feelings related to their experience that last long after the traumatic event has ended. They may relive the event through flashbacks or nightmares; they may feel sadness, fear or anger; and they may feel detached or estranged from other people. People with PTSD may avoid situations or people that remind them of the traumatic event, and they may have strong negative reactions to something as ordinary as a loud noise or an accidental touch. Symptoms of PTSD fall into the following four categories. Specific symptoms can vary in severity. Intrusion: Intrusive thoughts such as repeated, involuntary memories; distressing dreams; or flashbacks of the traumatic event. Flashbacks may be so vivid that people feel they are reliving the traumatic experience or seeing it before their eyes. Avoidance: Avoiding reminders of the traumatic event may include avoiding people, places, activities, objects and situations that may trigger distressing memories. People may try to avoid remembering or thinking about the traumatic event. They may resist talking about what happened or how they feel about it. Alterations in cognition and mood: Inability to remember important aspects of the traumatic event, negative thoughts and feelings leading to ongoing and distorted beliefs about oneself or others: distorted thoughts about the cause or consequences of the event leading to wrongly blaming self or other; ongoing fear, horror, anger, guilt or shame; much less interest in activities previously enjoyed; feeling detached or estranged from others; or being unable to experience positive emotions (a void of happiness or satisfaction). Symptoms of PTSD include alterations in arousal and reactivity: Arousal and reactive symptoms may include being irritable and having angry outbursts; behaving recklessly or in a self-destructive way; being overly watchful of one's surroundings in a suspecting way; being easily startled; or having problems concentrating or sleeping.

Many people who are exposed to a traumatic event experience symptoms similar to those described above in the days following the event. For a person to be diagnosed with PTSD, however, symptoms must last for more than a month and must cause significant distress or problems in the individual's daily functioning. Many individuals develop symptoms within three months of the trauma, but symptoms may appear later and often persist for months and sometimes years. PTSD often occurs with other related conditions, such as depression, substance use, memory problems and other physical and mental health problems. See https://www.psychiatry.org/patients-families/ptsd/what-is-ptsd, last accessed on Apr. 6, 2023.

Psychological and psychiatric support is used to treat PTSD as well as supportive drug therapy. Two medications used to treat PTSD in adults in paroxetine and sertraline as well as other selective serotonin reuptake inhibitors (SSRIs).

miRNAs or microRNAs are a class of small noncoding RNAs of about 22 nucleotides in length which are involved in the regulation of gene expression at the posttranscriptional level by degrading their target mRNAs and/or inhibiting their translation. Methods to isolate and quatify miRNA are known in the art, e.g., McAlexander et al. (2013) Frontiers Genetics, 16 May 2013, Vol. 4 see https://doi.org/10.3389/fgene.2013.00083; Monleau et al. (2014) BMC Genomics, Vol. 15, No.: 395, each last accessed on Apr. 6, 2023.

### Descriptive Embodiments

The disclosure further provides diagnostic, prognostic and therapeutic methods, which are based, at least in part, on determination of a selected miRNA profile isolated from a patient's biological sample, e.g., PBMCs.

For example, information obtained using the diagnostic assays described herein is useful for determining if a subject is suitable for TBI or mTBI. Based on the prognostic information, a doctor can recommend a therapeutic protocol, useful for treating TMI, mTBI or PTSD.

It is to be understood that information obtained using the diagnostic assays described herein can be used alone or in combination with other information, such as, but not limited to, genotypes or expression levels of other genes, clinical chemical parameters, histopathological parameters, clinical parameters, psychological tests, cognitive tests, or age, gender and weight of the subject. When used alone, the information obtained using the diagnostic assays described herein is useful in determining or identifying the clinical outcome of a treatment, selecting treatment for a patient, monitoring therapy, selecting a patient for a treatment, or treating a patient, etc. When used in combination with other information, on the other hand, the information obtained using the diagnostic assays described herein is useful in aiding in the determination or identification of clinical outcome of a treatment, aiding in the selection of a patient for a treatment, or aiding in the treatment of a patient and etc.

The methods are useful in the assistance of an animal, a mammal or yet further a human patient. For the purpose of illustration only, a mammal includes but is not limited to a human, a simian, a murine, a bovine, an equine, a porcine or an ovine subject.

### Biological Sample Collection and Preparation

Methods of obtaining test samples are known to those of skill in the art and include, but are not limited to, aspirations, tissue sections, swabs, drawing of blood or other fluids, surgical or needle biopsies.

In some aspects, the biological sample is a tissue or a cell sample. Suitable patient samples in the methods include, but are not limited to, blood, plasma, peripheral blood lymphocytes, serum, a biopsy tissue, fine needle biopsy sample, amniotic fluid, plasma, pleural fluid, saliva, semen, serum, tissue or tissue homogenates, frozen or paraffin sections of tissue or combinations thereof. In some aspects, the biological sample comprises, or alternatively consisting essentially of, or yet further consisting of, at least one of a tumor cell, a normal cell adjacent to a tumor, a normal cell corresponding to the tumor tissue type, a blood cell, a peripheral blood lymphocyte, or combinations thereof. In some aspects, the biological sample is an original sample recently isolated from the patient, a fixed tissue, a frozen tissue, a resection tissue, or a microdissected tissue. In some aspects, the biological samples are processed, such as by sectioning of tissues, fractionation, purification, nucleic acid isolation, or cellular organelle separation.

In some embodiments, nucleic acid (DNA or RNA) is isolated from the sample according to any methods known to those of skill in the art. In some aspects, genomic DNA is isolated from the biological sample. In some aspects, RNA is isolated from the biological sample. In some aspects, cDNA is generated from mRNA in the sample. In some embodiments, the nucleic acid is not isolated from the biological sample (e.g., the polymorphism is detected directly from the biological sample).

In another embodiment of the disclosure, the plurality of miRNA are capable of hybridizing specifically to the nucleic acid containing the allelic variant are attached to a solid phase support, e.g., a “chip” or “microarray. Such gene chips or microarrays can be used to detect genetic variations by a number of techniques known to one of skill in the art. In one technique, oligonucleotides are arrayed on a gene chip for determining the DNA sequence by the sequencing by hybridization approach. The probes of the disclosure also can be used for fluorescent detection of a genetic sequence. A probe also can be affixed to an electrode surface for the electrochemical detection of nucleic acid sequences.

In one aspect, “gene chips” or “microarrays” containing probes or primers for the gene of interest are provided alone or in combination with other probes and/or primers. A suitable sample is obtained from the patient extraction of genomic DNA, RNA, or any combination thereof and amplified if necessary. The DNA or RNA sample is contacted to the gene chip or microarray panel under conditions suitable for hybridization of the gene(s) of interest to the probe(s) or primer(s) contained on the gene chip or microarray. The probes or primers can be detectably labeled thereby identifying the polymorphism in the gene(s) of interest. Alternatively, a chemical or biological reaction can be used to identify the probes or primers which hybridized with the DNA or RNA of the gene(s) of interest. The genetic profile of the patient is then determined with the aid of the aforementioned apparatus and methods.

In some aspects, whole genome sequencing, in particular with the “next generation sequencing” techniques, which employ massively parallel sequencing of DNA templates, can be used to obtain genotypes of relevant polymorphisms. Exemplary NGS sequencing platforms for the generation of nucleic acid sequence data include, but are not limited to, Illumina's sequencing by synthesis technology (e.g., Illumina MiSeq or HiSeq System), Life Technologies' Ion Torrent semiconductor sequencing technology (e.g., Ion Torrent PGM or Proton system), the Roche (454 Life Sciences) GS series and Qiagen (Intelligent BioSystems) Gene Reader sequencing platforms.

Detectable labels can be used to identify the primer or probe hybridized to a genomic nucleic acid or amplicon. Detectable labels include but are not limited to fluorophores, isotopes (e.g., ³²P, ³³P, ³⁵S, ³H, ¹⁴C, ¹²⁵I, ¹³¹I) electron-dense reagents (e.g., gold, silver), nanoparticles, enzymes commonly used in an ELISA (e.g., horseradish peroxidase, beta-galactosidase, luciferase, alkaline phosphatase), chemiluminescent compounds, colorimetric labels (e.g., colloidal gold), magnetic labels (e.g., Dynabeads®), biotin, digoxigenin, haptens, proteins for which antisera or monoclonal antibodies are available, ligands, hormones, oligonucleotides capable of forming a complex with the corresponding oligonucleotide complement.

In one embodiment, a primer or probe is labeled with a fluorophore that emits a detectable signal. The term “fluorophore” as used herein refers to a molecule that absorbs light at a particular wavelength (excitation frequency) and subsequently emits light of a longer wavelength (emission frequency). While a suitable reporter dye is a fluorescent dye, any reporter dye that can be attached to a detection reagent such as an oligonucleotide probe or primer is suitable for use in the methods described. Suitable fluorescent moieties include, but are not limited to, the following fluorophores working individually or in combination: 4-acetamido-4′-isothiocyanatostilbene-2,2′disulfonic acid; acridine and derivatives, e.g., acridine, acridine isothiocyanate; Alexa Fluors: Alexa Fluor® 350, Alexa Fluor® 488, Alexa Fluor® 546, Alexa Fluor® 555, Alexa Fluor® 568, Alexa Fluor® 594, Alexa Fluor® 647 (Molecular Probes); 5-(2′-aminoethyl)aminonaphthalene-1-sulfonic acid (EDANS); 4-amino-N-[3-vinylsulfonyl)phenyl]naphthalimide-3,5 disulfonate (Lucifer Yellow VS); N-(4-anilino-1-naphthyl)maleimide; anthranilamide; Black Hole Quencher™ (BHQ™) dyes (biosearch Technologies); BODIPY dyes: BODIPY® R-6G, BOPIPY® 530/550, BODIPY® FL; Brilliant Yellow; coumarin and derivatives: coumarin, 7-amino-4-methylcoumarin (AMC, Coumarin 120), 7-amino-4-trifluoromethylcouluarin (Coumarin 151); Cy2®, Cy3®, Cy3.5®, Cy5®, Cy5.5®; cyanosine; 4′,6-diaminidino-2-phenylindole (DAPI); 5′,5″-dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); Eclipse™ (Epoch Biosciences Inc.); eosin and derivatives: eosin, eosin isothiocyanate; erythrosin and derivatives: erythrosin B, erythrosin isothiocyanate; ethidium; fluorescein and derivatives: 5-carboxyfluorescein (FAM), 5-(4,6-dichlorotriazin-2-yl)aminofluorescein (DTAF), 2′,7′-dimethoxy-4′5′-dichloro-6-carboxyfluorescein (JOE), fluorescein, fluorescein isothiocyanate (FITC), hexachloro-6-carboxyfluorescein (HEX), QFITC (XRITC), tetrachlorofluorescein (TET); fluorescamine; IR144; IR1446; lanthamide phosphors; Malachite Green isothiocyanate; 4-methylumbelliferone; ortho cresolphthalein; nitrotyrosine; pararosaniline; Phenol Red; B-phycoerythrin, R-phycoerythrin; allophycocyanin; o-phthaldialdehyde; Oregon Green®; propidium iodide; pyrene and derivatives: pyrene, pyrene butyrate, succinimidyl 1-pyrene butyrate; QSY® 7; QSY® 9; QSY® 21; QSY® 35 (Molecular Probes); Reactive Red 4 (Cibacron® Brilliant Red 3B-A); rhodamine and derivatives: 6-carboxy-X-rhodamine (ROX), 6-carboxyrhodamine (R6G), lissamine rhodamine B sulfonyl chloride, rhodamine (Rhod), rhodamine B, rhodamine 123, rhodamine green, rhodamine X isothiocyanate, riboflavin, rosolic acid, sulforhodamine B, sulforhodamine 101, sulfonyl chloride derivative of sulforhodamine 101 (Texas Red); terbium chelate derivatives; N,N,N′,N′-tetramethyl-6-carboxyrhodamine (TAMRA); tetramethyl rhodamine; and tetramethyl rhodamine isothiocyanate (TRITC).

In some aspects, the primer or probe is further labeled with a quencher dye such as Tamra, Dabcyl, or Black Hole Quencher® (BHQ), especially when the reagent is used as a self-quenching probe such as a TaqMan® (U.S. Pat. Nos. 5,210,015 and 5,538,848) or Molecular Beacon probe (U.S. Pat. Nos. 5,118,801 and 5,312,728), or other stemless or linear beacon probe (Livak et al., 1995, PCR Method Appl., 4:357-362; Tyagi et al, 1996, Nature Biotechnology, 14:303-308; Nazarenko et al., 1997, Nucl. Acids Res., 25:2516-2521; U.S. Pat. Nos. 5,866,336 and 6,117,635).

In some aspects, methods for real time PCR use fluorescent primers/probes, such as the TaqMan® primers/probes (Heid, et al., Genome Res 6: 986-994, 1996), molecular beacons, and Scorpion™ primers/probes. Real-time PCR quantifies the initial amount of the template with more specificity, sensitivity and reproducibility, than other forms of quantitative PCR, which detect the amount of final amplified product. Real-time PCR does not detect the size of the amplicon. The probes employed in Scorpion®™ and TaqMan® technologies are based on the principle of fluorescence quenching and involve a donor fluorophore and a quenching moiety. The term “donor fluorophore” as used herein means a fluorophore that, when in close proximity to a quencher moiety, donates or transfers emission energy to the quencher. As a result of donating energy to the quencher moiety, the donor fluorophore will itself emit less light at a particular emission frequency that it would have in the absence of a closely positioned quencher moiety. The term “quencher moiety” as used herein means a molecule that, in close proximity to a donor fluorophore, takes up emission energy generated by the donor and either dissipates the energy as heat or emits light of a longer wavelength than the emission wavelength of the donor. In the latter case, the quencher is considered to be an acceptor fluorophore. The quenching moiety can act via proximal (i.e., collisional) quenching or by Forster or fluorescence resonance energy transfer (“FRET”). Quenching by FRET is generally used in TaqMan® primers/probes while proximal quenching is used in molecular beacon and Scorpion™ type primers/probes.

The detectable label can be incorporated into, associated with or conjugated to a nucleic acid primer or probe. Labels can be attached by spacer arms of various lengths to reduce potential steric hindrance or impact on other useful or desired properties. See, e.g., Mansfield, Mol. Cell. Probes (1995), 9:145-156.

Detectable labels can be incorporated into nucleic acid probes by covalent or non-covalent means, e.g., by transcription, such as by random-primer labeling using Klenow polymerase, or nick translation, or, amplification, or equivalent as is known in the art. For example, a nucleotide base is conjugated to a detectable moiety, such as a fluorescent dye, e.g., Cy3™ or Cy5™ and then incorporated into nucleic acid probes during nucleic acid synthesis or amplification. Nucleic acid probes can thereby be labeled when synthesized using Cy3™- or Cy5™-dCTP conjugates mixed with unlabeled dCTP.

Nucleic acid probes can be labeled by using PCR or nick translation in the presence of labeled precursor nucleotides, for example, modified nucleotides synthesized by coupling allylamine-dUTP to the succinimidyl-ester derivatives of the fluorescent dyes or haptens (such as biotin or digoxigenin) can be used; this method allows custom preparation of most common fluorescent nucleotides, see, e.g., Henegariu et al., Nat. Biotechnol. (2000), 18:345-348,

Nucleic acid probes can be labeled by non-covalent means known in the art. For example, Kreatech Biotechnology's Universal Linkage System® (ULS®) provides a non-enzymatic labeling technology, wherein a platinum group forms a co-ordinative bond with DNA, RNA or nucleotides by binding to the N7 position of guanosine. This technology can also be used to label proteins by binding to nitrogen and sulfur containing side chains of amino acids. See, e.g., U.S. Pat. Nos. 5,580,990; 5,714,327; and 5,985,566; and European Patent No. 0539466.

Labeling with a detectable label also can include a nucleic acid attached to another biological molecule, such as a nucleic acid, e.g., an oligonucleotide, or a nucleic acid in the form of a stem-loop structure as a “molecular beacon” or an “aptamer beacon”. Molecular beacons as detectable moieties are described; for example, Sokol (Proc. Natl. Acad. Sci. USA (1998), 95:11538-11543) synthesized “molecular beacon” reporter oligodeoxynucleotides with matched fluorescent donor and acceptor chromophores on their 5′ and 3′ ends. In the absence of a complementary nucleic acid strand, the molecular beacon remains in a stem-loop conformation where fluorescence resonance energy transfer prevents signal emission. On hybridization with a complementary sequence, the stem-loop structure opens increasing the physical distance between the donor and acceptor moieties thereby reducing fluorescence resonance energy transfer and allowing a detectable signal to be emitted when the beacon is excited by light of the appropriate wavelength. See also, e.g., Antony (Biochemistry (2001), 40:9387-9395), describing a molecular beacon consist of a G-rich 18-mer triplex forming oligodeoxyribonucleotide. See also U.S. Pat. Nos. 6,277,581 and 6,235,504.

Aptamer beacons are similar to molecular beacons; see, e.g., Hamaguchi, Anal. Biochem. (2001), 294:126-131; Poddar, Mol. Cell. Probes (2001), 15:161-167; Kaboev, Nucleic Acids Res. (2000), 28:E94. Aptamer beacons can adopt two or more conformations, one of which allows ligand binding. A fluorescence-quenching pair is used to report changes in conformation induced by ligand binding. See also, e.g., Yamamoto et al., Genes Cells (2000), 5:389-396; Smimov et al., Biochemistry (2000), 39:1462-1468.

The nucleic acid primer or probe can be indirectly detectably labeled via a peptide. A peptide can be made detectable by incorporating predetermined polypeptide epitopes recognized by a secondary reporter (e.g., leucine zipper pair sequences, binding sites for secondary antibodies, transcriptional activator polypeptide, metal binding domains, epitope tags). A label can also be attached via a second peptide that interacts with the first peptide (e.g., S—S association).

As readily recognized by one of skill in the art, detection of the complex containing the nucleic acid from a sample hybridized to a labeled probe can be achieved through use of a labeled antibody against the label of the probe. In one example, the probe is labeled with digoxigenin and is detected with a fluorescent labeled anti-digoxigenin antibody. In another example, the probe is labeled with FITC, and detected with fluorescent labeled anti-FITC antibody. These antibodies are readily available commercially. In another example, the probe is labeled with FITC, and detected with anti-FITC antibody primary antibody and a labeled anti-anti FITC secondary antibody.

Nucleic acids can be amplified prior to detection or can be detected directly during an amplification step (i.e., “real-time” methods, such as in TaqMan® and Scorpion™ methods). In some embodiments, the target sequence is amplified using a labeled primer such that the resulting amplicon is detectably labeled. In some embodiments, the primer is fluorescently labeled. In some embodiments, the target sequence is amplified and the resulting amplicon is detected by electrophoresis.

With regard to the exemplary primers and probes, those skilled in the art will readily recognize that nucleic acid molecules can be double-stranded molecules and that reference to a particular site on one strand refers, as well, to the corresponding site on a complementary strand. In defining a variant position, allele, or nucleotide sequence, reference to an adenine, a thymine (uridine), a cytosine, or a guanine at a particular site on one strand of a nucleic acid molecule also defines the thymine (uridine), adenine, guanine, or cytosine (respectively) at the corresponding site on a complementary strand of the nucleic acid molecule. Thus, reference can be made to either strand in order to refer to a particular variant position, allele, or nucleotide sequence. Probes and primers, can be designed to hybridize to either strand and detection methods disclosed herein can generally target either strand.

In some embodiments, the primers and probes comprise additional nucleotides corresponding to sequences of universal primers (e.g., T7, M13, SP6, T3) which add the additional sequence to the amplicon during amplification to permit further amplification and/or prime the amplicon for sequencing.

### Diagnostic Methods

Applicant provides several methods in this disclosure, each comprising, or consisting essentially of, or yet further consisting of analyzing a plurality of miRNA isolated from a biological sample isolated from the patient by a method comprising, or consisting essentially of, or yet further consisting of the method disclosed herein. In one aspect, the method provides for selecting a patient for traumatic brain injury (TBI) therapy. In this method, the plurality of miRNA is isolated an analyzed according to the method, and a score which satisfies a threshold selects the patient for the TBI therapy. The threshold may be determined or derived using various machine learning models (such as a support vector machine, decision tree, random forest, etc.), may be trained and modified over time based on a feedback algorithm which compares an actual patient diagnosis to a predicted diagnosis using an output from one of the diagnosis methods described herein, and so forth. Examples of TBI therapy are provided infra, and incorporated by reference herein. If the score does not satisfy the threshold, the patient can undergo further analysis or treatment for PTSD. In one aspect, the patient is a mammal, e.g. a human patient. In a further aspect, the patient is exhibiting cognitive symptoms of TBI, mTBI and/or PTSD. A physical, patient or other healthcare professional can use the disclosed method to diagnose the patient and/or select the best therapy for treatment.

In some embodiments, the patient can be tested for TBI using a PCR test (such as a quick or real-time PCR test). The PCR test may identify expression levels of certain miRNA present in the patient's sample. The PCR test may include various probes which are configured to measure, detect, or otherwise identify the expression levels of the miRNA present in the patient's sample. The PCR test may include probes which measure, detect, or otherwise identify expression levels of, for example, 18 miRNA nucleic acid sequences. The PCR test may include probes which identify expression levels of the following miRNA listed in Table 1.

TABLE 1
Targeted nucleic acid sequences for PCR analysis
miRNA

| | | |
|---|---|---|
| U8 | U58B | U27 |
| U83A | HBII-289 | U55 |
| HBII-239 | U38B | U56 |
| U15B | U35A | ACA48 |
| U91 | ENSG000001941 | hsa-miR-671-5p |
| hcmv-miR-US4 | hsa-miR1285 | hsa-miR455-3p |

The miRNA included in Table 1 may be biomarkers for selective probe systems for PCR tests. The miRNA may have been identified using one or more of the methodologies described in Pasinetti et al., “Select non-coding RNA in blood components provide novel clinically accessible biological surrogates for improved identification of traumatic brain injury in OEF/OIF Veterans,” National Institute of Health, pp. 88-98, published May 30, 2012, the contents of which are incorporated herein by reference in their entirety.

To determine subgroups for identifying the small RNA from the miRNA sequences included in Table 1, the system may normalize the PCR dCT values and store in a new layer of an expression matrix (e.g., ExpressMatrix2, Offset: Constant Value=0, Scaling: Constant Value=−1). The system may then normalize the expression matrix (e.g., ExpressMatrix2), and store in another new layer (GN_M1, Offset: Mean/Average, Scaling: Constant Value=1). The system may define groupings based on patient types and conditions (e.g., TBI with PTSD, TBI without PTSD, Non-TBI with PTSD, Non-TBI without PTSD). The system may establish two new subsets based on the defined groupings (e.g., Co-morbid PTSD groups, or TBI with PTSD/Non-TBI with PTSD, and non-PTSD groups, or TBI without PTSD/Non-TBI without PTSD). The system may filter the two subsets (e.g., for T-Test p-value being less than or equal to 0.05), and create new subsets. Within each subgroup, the system may select half of the samples from each group, and create a training set subgroup with an equal number of members from each group. The system may create or establish subgroups (each of which have the same number of members from each group—e.g., four TBI with PTSD, four nonTBI with PTSD) with permutations of various combinations of the small RNA sequences from Table 1 (or elsewhere) (e.g., ACA48/U35A/U55, ACA48/U35A/U83A, U35A/U55/U83A, U35A/U83A, etc.).

Once the system has subgroups with permutations of various combinations of small RNA sequences, together with filtered and normalized data from the PCR tests of samples, the system may perform or execute a clustering algorithm (e.g., using unweighted pair group method with arithmetic mean clustering, or some other clustering algorithm) to identify a method or grouping with the highest accuracy for each permutation. The system may train a classifier (such as a support vector machine or some other classifier) based on the clustered data for each permutation. The system may generate classification tables (e.g., a spreadsheet showing the training set, results, labels, etc.) for each grouping and permutation. The system may perform receiver operating characteristics (ROC) analysis using the classification tables, to indicate the efficacy of various classifiers in classifying TBI vs. non-TBI with or without PTSD co-morbidity (e.g., higher AUC=better classifier). The system may select a permutation having a strongest classification output (e.g., higher AUC). The permutation selected by the system may be a combination of ACA48, U35A, U55, and U83A (though it is noted that other permutations any be used).

In some embodiments, the system may compute, determine, or otherwise establish the threshold against which an miRNA score is compared. For example, the system may use a support vector machine for determining a hyperplane against which the score is compared. The system may train the support vector machine using similar data as described above, to determine the optimal hyperplane. The optimal hyperplane may be or define a threshold value between 0 and 1, against which the miRNA score is compared. While described as using a support vector machine, it is noted that the threshold may be determined using a logistic regression, decision tree or random forest, or other classification models which are configured or designed to generate or determine thresholds for different classification modeling.

miRNA Scoring

In some embodiments, the classification algorithm may also receive inputs indicative of various clinical factors of each patient. The clinical factors may include, for example, gender, whether the patient has been diagnosed with PTSD, a time from traumatic event, an age, etc. The system may generate or develop coorelates by selecting from the microarray profiles for each clinical factor the top-ranked RNAs (mRNAs and sncRNA) that differentiate each of these clinical characteristics, based on expression levels and fitting them to a model using logistic regression. RNAs may be selected using the entire training set and each correlate is based on a minimal set of RNAs to represent the clinical factor as accurately as possible (typically 2 to 12 RNAs). In some embodiments, the system may develop one or more models using logistic regression of mTBI status (0/1) on 1) gender, 2) PTSD status (PCL-C score), 3) time from last deployment (or from traumatic event, if known) (yrs), and on age (yrs). For each clinical factor model (CFM), the system can screen RNAs based on an empirical Bayes t-test. The system can include the top RNAs, selected based on the p-value and fold change, in a logistic regression model where the relevant clinical factor is the dependent variable. Resulting values derived from each CFM are converted to a prediction score ranging between 0 and 1, where CFM score is equal to a sigmoid function, or exp(x)/(1+exp(x)) for individual testing.

To estimate the final model coefficients, a penalized logistic regression model can be used, with genomic gender, genomic PTSD status, genomic time from deployment/traumatic event, age, and the final reduced RNA cluster means as the independent predictors, and mTBI status (0/1) as the dependent variable. Penalization factors (lambda) can be determined for each of the clinical/genomic correlates and each of the mTBI RNA expression clusters.

The final classification algorithm (e.g., assuming a support vector machine classification algorithm) may be defined as:

$$X = sum ( {W}_{0} + {W}_{i} * {F}_{i} )$$

where, X is the logistic regression score, Wi are coefficients, and Fi are the values for each of the features. The features may include “genomic gender”, “genomic PTSD status”, “genomic time from deployment/traumatic event”, and “age” or “genomic age”, along with mTBI RNAs (represented as the mean of each final mTBI RNA expression cluster); Wo is the intercept. The logistic regression score can be converted to a prediction score using a sigmoid function.

In some embodiments, a system of the present solution may analyze the output from a PCR system testing for the small RNA sequences to compute or determine a likelihood/score/value of the patient having TBI as compared to one or more alternative conditions (such as PTSD). The system may include a processing or computer system including any number of processors and memory, where the processors are separately or collectively configured to analyze the output from the PCR system according to various models. The PCR system may be configured to output PCR dCT (delta cycle threshold) values for each marker of the sample. The system may compute an average of the PCR dCT values for each marker for the sample(s) and remove/filter values from calculations based on a coefficient of variance being greater than a certain value (e.g., greater than or equal to 0.1). The system may then apply the filtered values for each marker to the classification model, along with other features of the patient (e.g., genomic gender, genomic PTSD status, genomic time from traumatic event, age, etc.), to the classification model, to compute a first value (e.g., the logistic regression score. The system may then compute a prediction score (e.g., using a sigmoid function) based on the logistic regression score for the patient. The system may compare the prediction score to a threshold. Based on the comparison, the system may determine whether the patient has PTSD and/or TBI. For example, if the prediction score is greater than the threshold, the system may determine that the patient has TBI. Similarly, if the prediction score is less than the threshold, the system may determine that the patient has PTSD.

Additionally, the system may track a progression of treatment for TBI based on multiple tests over time. For example, the system may compute a first prediction score at a first time instance (such as at diagnosis), and a second prediction score at a second time instance (e.g., while the patient is undergoing treatment for TBI using one or more of the methods described herein, or other methods for treating TBI). The system may compare the first prediction score to the second prediction score. The system may determine a treatment efficacy based on the comparison. For example, the system may suggest alternative treatments where the first prediction score is substantially the same as the second prediction score (e.g., indicating nominal improvement to no improvement).

### Methods of Treatment

The disclosure further provides methods of treating a patient selected by any method of the above embodiments, or identified as likely to experience a more favorable clinical outcome by any of the above methods, following the therapy. In some embodiments, the methods entail administering to the patients such a therapy. Such therapies may include, for example, administering pharmaceutical compositions, surgery to reduce intracranial hemorrhage or reduce pressure in brain swelling, rehabilitation therapies, rest, counseling, etc.

TBI treatments are known in the art, and examples of such are provided infra and incorporated herein by reference. In one aspect, the patient is a human patient. In another aspect, the patient is suffering from symptoms of TBI. In one aspect, the the biological sample comprises peripheral blood mononuclear cells.

Methods of administering pharmaceutical compositions are well known to those of ordinary skill in the art and include, but are not limited to, oral, microinjection, intravenous or parenteral administration. The compositions are intended for topical, oral, or local administration as well as intravenously, subcutaneously, or intramuscularly. Administration can be effected continuously or intermittently throughout the course of the treatment. Methods of determining the most effective means and dosage of administration are well known to those of skill in the art and will vary with the disease or injury being being treated and the patient and the subject being treated. Single or multiple administrations can be carried out with the dose level and pattern being selected by the treating physician.

### Kits

Kits or panel for use in detecting the plurality of miRNA in patient biological samples are provided such as Table 1. In some embodiments, a kit comprises at least one reagent necessary to perform the assay. For example, the kit can comprise an enzyme, a buffer or any other necessary reagent (e.g. PCR reagents and buffers). For example, in some aspects, a kit contains, in an amount sufficient for at least one assay, any of the hybridization assay probes, amplification primers, and/or antibodies suitable for detection in a packaging material. In some embodiments, the kit or panel comprises primer and/or probes suitable for screening for the plurality of miRNA.

In some aspects, the kit or panel is for selecting therapy for a TBI patient or monitoring therapy of a TMI patient. In some aspects, the kit or panel is for determining the eligibility of a patient for TMI or PTSD therapy, for example those identified herein.

Typically, the kits will also include instructions recorded in a tangible form (e.g., contained on paper or an electronic medium) for using the packaged probes, primers, and/or antibodies in a detection assay for determining the presence or amount of the plurality of miRNA in a test sample.

In some aspects, the kits further comprise a solid support for anchoring the nucleic acid of interest on the solid support. The target nucleic acid can be anchored to the solid support directly or indirectly through a capture probe anchored to the solid support and capable of hybridizing to the nucleic acid of interest. Examples of such solid support include but are not limited to beads, microparticles (for example, gold and other nano particles), microarray, microwells, multiwell plates. The solid surfaces can comprise a first member of a binding pair and the capture probe or the target nucleic acid can comprise a second member of the binding pair. Binding of the binding pair members will anchor the capture probe or the target nucleic acid to the solid surface. Examples of such binding pairs include but are not limited to biotin/streptavidin, hormone/receptor, ligand/receptor, and antigen/antibody.

The test samples used in the diagnostic kits include cells, protein or membrane extracts of cells, or biological fluids such as sputum, blood, PMBCs, serum, plasma, or urine. The test samples can also be a tumor cell, a normal cell adjacent to a tumor, a normal cell corresponding to the tumor tissue type, a blood cell, a peripheral blood lymphocyte, or combinations thereof. The test sample used in the above-described method will vary based on the assay format, nature of the detection method and the tissues, cells or extracts used as the sample to be assayed. Methods for preparing miRNA extracts are known in the art and can be readily adapted in order to obtain a sample which is compatible with the system utilized.

The kits can include all or some of the positive controls, negative controls, reagents, primers, sequencing markers, probes and antibodies described herein for determining the subject's genotype in the polymorphic region of the gene of interest or target region.

As amenable, these suggested kit components can be packaged in a manner customary for use by those of skill in the art. For example, these suggested kit components can be provided in solution or as a liquid dispersion or the like.

Typical packaging materials would include solid matrices such as glass, plastic, paper, foil, micro-particles and the like, capable of holding within fixed limits hybridization assay probes, and/or amplification primers. Thus, for example, the packaging materials can include glass vials used to contain sub-milligram (e.g., picogram or nanogram) quantities of a contemplated probe, primer, or antibodies or they can be microtiter plate wells to which probes, primers, or antibodies have been operatively affixed, i.e., linked so as to be capable of participating in an amplification and/or detection methods.

The instructions will typically indicate the reagents and/or concentrations of reagents and at least one assay method parameter which might be, for example, the relative amounts of reagents to use per amount of sample. In addition, such specifics as maintenance, time periods, temperature, and buffer conditions can also be included.

The disclosure now being generally described, it will be more readily understood by reference to the following example which is included merely for purposes of illustration of certain aspects and embodiments of the present disclosure, and are not intended to limit the disclosure.

### EQUIVALENTS

The disclosure illustratively described herein can suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed.

Thus, it should be understood that although the present disclosure has been specifically disclosed by preferred embodiments and optional features, modification, improvement and variation of the disclosure embodied therein herein disclosed can be resorted to by those skilled in the art, and that such modifications, improvements and variations are considered to be within the scope of this disclosure. The materials, methods, and examples provided here are representative of preferred embodiments, are exemplary, and are not intended as limitations on the scope of the disclosure.

The disclosure has been described broadly and generically herein. Each of the narrower species and subgeneric groupings falling within the generic disclosure also form part of the disclosure. This includes the generic description of the disclosure with a proviso or negative limitation removing any subject matter from the genus, regardless of whether or not the excised material is specifically recited herein.

In addition, where features or aspects of the disclosure are described in terms of Markush groups, those skilled in the art will recognize that the disclosure is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

What is claimed is:

1. A method for selecting a patient for traumatic brain injury (TBI) therapy including analyzing a plurality of miRNA isolated from a biological sample isolated from the patient by a method comprising:
maintaining a classification model configured to generate an output which is a function of expression levels of the plurality of miRNA, one or more clinical factors, and one or more offsets or weights;
receiving data indicative of expression levels of the plurality of miRNA from a plurality of respective probes of a polymerase chain reaction (PCR) test of the patient;
computing a first score as an output from the classification model, based on the one or more clinical factors for the patient and the expression levels of the plurality of miRNA in the biological sample; and
computing a second score based on the first score, the second score being within a range of values;
wherein the second score satisfies a threshold value within the range selects the patient for the TBI therapy.

2. The method of claim 1, wherein the one or more clinical factors comprise genomic gender, genomic post-traumatic stress disorder (PTSD) status, genomic time from event, and age.

3. The method of claim 1, wherein the classification model comprises a support vector machine with a slope being a coefficient, an offset as the intercept, and dependent variables as values for the one or more clinical values and expression levels of the subset of miRNAs.

4. The method of claim 3, wherein the second score is computed as a sigmoid function of the first score from the classification model.

5. The method of claim 1, further comprising training the classification model using a training dataset, to determine the one or more offsets or weights.

6. The method of claim 1, wherein the plurality of respective probes are configured to measure expression levels of the plurality of miRNA, the plurality of miRNA comprising U8, U58B, U27, U83A, HBII-289, U55, HBII-239, U38B, U56, U15B, U35A, ACA48, U91, ENSG000001941, hsa-miR-671-5p, hcmv-miR-US4, hsa-miR1285, or hsa-miR455-3p.

7. A method for selecting a patient for post traumatic stress disorder (PTSD) therapy including analyzing a plurality of miRNA isolated from a biological sample isolated from the patient by a method comprising:
maintaining a classification model configured to generate an output which is a function of expression levels of the plurality of miRNA, one or more clinical factors, and one or more offsets or weights;
receiving data indicative of expression levels of the plurality of miRNA from a plurality of respective probes of a polymerase chain reaction (PCR) test of the patient;
computing a first score as an output from the classification model, based on the one or more clinical factors for the patient and the expression levels of the plurality of miRNA in the biological sample; and computing a second score based on the first score, the second score being within a range of values;
wherein a score which satisfies a threshold value within the range of values selects the patient for the PTSD therapy.

8. A method for determining if a patient is suffering from TBI or PTSD including analyzing a plurality of miRNA isolated from a biological sample isolated from the patient by a method comprising:
maintaining a classification model configured to generate an output which is a function of expression levels of the plurality of miRNA, one or more clinical factors, and one or more offsets or weights;
receiving data indicative of expression levels of the plurality of miRNA from a plurality of respective probes of a polymerase chain reaction (PCR) test of the patient;
computing a first score as an output from the classification model, based on the one or more clinical factors for the patient and the expression levels of the plurality of miRNA in the biological sample; and
computing a second score based on the first score, the second score being within a range of values;
wherein a score greater than a threshold value identifies the patient is suffering from TBI and a score less than the threshold value identifies the patient as suffering from PTSD.

9. A method for monitoring TBI therapy and determining if a patient receiving TBI therapy is improving, comprising:
maintaining a classification model configured to generate an output which is a function of expression levels of the plurality of miRNA, one or more clinical factors, and one or more offsets or weights;
analyzing a first plurality of miRNA isolated from a biological sample isolated from the patient who has received TBI therapy by a method comprising:
receiving data indicative of first expression levels of the plurality of miRNA from a plurality of respective probes of a first polymerase chain reaction (PCR) test of the patient;
computing a first score as an output from the classification model, based on the one or more clinical factors for the patient and the first expression levels of the plurality of miRNA in the biological sample; and
computing a second score based on the first score, the second score being within a range of values;
analyzing a second plurality of miRNA isolated from a biological sample isolated from the patient who has received TBI therapy by a method comprising:
receiving data indicative of second expression levels of the plurality of miRNA from a second PCR test of the patient;
computing a third score as an output from the classification model, based on the one or more clinical factors for the patient and the second expression levels of the plurality of miRNA in the biological sample; and
computing a fourth score based on the third score, the third score being within a range of values;
comparing the second score from the first plurality to the fourth score of the second plurality, wherein the fourth score being less than the second score identifies the patient as improving from the therapy.

10. A method for treating a patient selected for TBI treatment based on the presence of a score from a method comprising:
analyzing a plurality of miRNA isolated from a biological sample isolated from the patient by a method comprising:
receiving data indicative of expression levels of the plurality of miRNA from a plurality of respective probes of a first polymerase chain reaction (PCR) test of the patient;
computing a first score as an output from the classification model, based on the one or more clinical factors for the patient and the expression levels of the plurality of miRNA in the biological sample; and
computing a second score based on the first score, the second score being within a range of values.

11. The method of any preceding claim, wherein the patient is a human patient.

12. The method of any preceding claim, wherein the patient is suffering from symptoms of PTSD.

13. The method of any preceding claim, wherein the biological sample comprises peripheral blood mononuclear cells.
